(19) United States
(12) Patent Application Publication
(10) Pub. No.: US 2022/0008259 A1
(43) Pub. Date: Jan. 13, 2022

(54) Cotton swab fluffing device

(71) Applicant: Amer F. Qandah, Chicago, IL (US)

(72) Inventor: Amer F. Qandah, Chicago, IL (US)

(21) Appl. No.: 17/349,881

(22) Filed: Jun. 16, 2021

Related U.S. Application Data
(60) Provisional application No. 63/051,378, filed on Jul. 13, 2020.

(51) Int. Cl. A61F 13/38

(52) U.S. Cl. CPC A61F 13/385

(57) ABSTRACT

A fluffing device for cotton swabs has a first body having a central opening admitting a cotton end on a rod of a cotton swab, geared elements within the body engaged to rotate relative to one another, needles attached to individual ones of the geared elements, the needles positioned to penetrate the cotton end of the cotton swab with the swab inserted into the central opening, and a physical input constrained to rotate the geared elements back and forth through a limited arc. The geared elements rotating move the needles in a manner urging portions of the cotton swab away from the rod, expanding and fluffing the cotton end.

### CROSS-REFERENCE TO RELATED APPLICATIONS

The present application claims priority to U.S. provisional patent application No. 63/051,378, filed on Jul. 13, 2020. The entire disclosure is included herein in its entirety at least by reference.

### BACKGROUND OF THE INVENTION

### 1. Field of the Invention

The present invention is in the field of consumer products/cosmetics and pertains particularly to cotton swabs fluffing devices.

### 2. Discussion of the State of the Art

Standard cotton swabs (commonly known as Q-tips, or cotton buds in other parts of the world) can be found nearly in every modern home in package for everyday use. Cosmetic supplies such as makeup kits include cotton swabs, and so do first aid kits, various painting sets, sculpting, arts and other hygienic kits, just to mention a few examples.

Some of the most wished-for cotton swabs on Ebay.com and amazon.com are the expensive, fluffy, low count packages, while the most purchased are the high count, tightly packaged cheaper brands.

Tightly packaged cotton swabs are sold in bulky boxes causing cotton to compress and lose its most valued features such as being soft on the skin, uniform in shape, absorbent and fluffy.

Additionally, Packages of 100's of cotton swabs are used over periods that can stretch for months, if not more. Over this period cotton becomes compressed and riddled with pressure marks that can be harsh on the skin and less efficient.

Many packages of cotton swabs can be found with compressed cotton even before they reach consumers.

The alternative brands of low count packages, with a little more cotton and more room in the package to prevent compression, come with a higher price tag. The price can be as high as 10 times that of the store and more affordable brands.

Many people fluff their cotton swabs using their fingernails, which can be time consuming, leads to a broken nail or causes severe structural damage to the cotton.

Currently there are no devices in the art to compare present invention to.

Therefore, what is clearly needed is a cotton swab fluffing device that can bridge the gap between expensive and cheap brands, solving the problems mentioned above and providing consumers with a choice to save money and obtain more useful, softer and more absorbent cotton swabs. Such device would be the first of its kind since there are currently no known comparable devices.

### SUMMARY OF THE INVENTION

In one embodiment of the invention a fluffing device for cotton swabs is provided, comprising a first body having a central opening admitting a cotton end on a rod of a cotton swab, geared elements within the body engaged to rotate relative to one another, needles attached to individual ones of the geared elements, the needles positioned to penetrate the cotton end of the cotton swab with the swab inserted into the central opening, and a physical input constrained to rotate the geared elements back and forth through a limited arc. The geared elements rotating move the needles in a manner urging portions of the cotton swab away from the rod, expanding and fluffing the cotton end.

In one embodiment the physical input has a second body engaging the first body rotationally, relative rotation between the first and the second body rotating the geared elements. Also, in one embodiment the first and the second body are cylindrical bodies rotating on a common axis. In one embodiment the relative rotation of the cylindrical bodies is opposed in both rotary directions by spring elements. And in one embodiment the needles are conical in shape.

In one embodiment the needles vary in diameter along a length. Also, in one embodiment the needles have surface roughness. Also, in one embodiment an outside surface of one or both of the first and the second body are roughened or corrugated improving grip. In one embodiment the needles are implemented on the gears in a manner to penetrate the cotton end from different directions. And in one embodiment the central opening is larger in diameter than the cotton end of the cotton swab.

In another aspect of the invention a method for fluffing a cotton swab is provided, comprising inserting a cotton end on a rod of a cotton swab into a central opening of a first body of a fluffing device, penetrating the cotton end by needles attached to individual ones of geared elements operating in a second body of the fluffing device, the geared elements engaged to rotate relative to one another, and rotating a physical input constrained to rotate the geared elements back and forth through a limited arc, the rotation of the geared elements manipulating the needles in a manner urging portions of the cotton swab away from the rod, expanding and fluffing the cotton end.

In one embodiment the method comprises engaging the first body by a second body rotationally, relative rotation between the first and the second body rotating the geared elements. Also, in one embodiment the method comprises rotating the first and the second cylindrical bodies on a common axis. In one embodiment the method comprises opposing the relative rotation of the cylindrical bodies in both rotary directions by spring elements. And in one embodiment the method comprises implementing the needles as conical in shape.

In one embodiment the method comprises implementing the needles to vary in diameter along a length. In one embodiment the method comprises implementing the needles with surface roughness. In one embodiment the method comprises implementing an outside surface of one or both of the first and the second body as roughened or corrugated, improving grip. In one embodiment the method comprises implementing the needles on the gears in a manner to penetrate the cotton end from different directions. And in one embodiment the method comprises making the central opening larger in diameter than the cotton end of the cotton swab.

### A BRIEF DESCRIPTION OF THE DRAWINGS

Embodiments of the invention will now be described, by way of example only, with reference to the accompanying drawings in which corresponding reference symbols indicate corresponding parts, and in which:

FIG. 1 is a sectioned rear perspective view of the cotton swab fluffing device showing various components according to an embodiment of present invention.

FIG. 2 is partial exploded side elevation view of FIG. 1.

FIG. 3 is a front view illustrating the alignment of a gears and incomplete gears around the central housing volume.

FIG. 4a depicts the idle position of the locking mechanism caused by a cuboid like protrusions on the internal gear wheel and a calculated track cut from the plate. FIG. 4b and FIG. 4c are depictions of the same locking mechanism at the two opposing ends of the track.

FIG. 5a is a depiction of an alignment of a plurality of needles, in the idle position. These needles are attached to the incomplete gear. FIG. 5b and FIG. 5c are depictions of the same plurality of needles during a clockwise and counterclockwise rotations of the internal gear wheel, respectively.

FIG. 6 is a top view of a non-rotating housing connector in one embodiment of present invention.

FIG. 7a and FIG. 7b are depictions of the detent mechanism during a clockwise rotation of the rotatable housing connector in a present embodiment of the invention.

FIG. 8 is an illustration of the movement of the various parts in a counterclockwise rotation of a housing connector, according to one example of present invention.

### DETAILED DESCRIPTION OF THE INVENTION

The problem stated above is that fluffed cotton swabs are desirable in most everyday applications. But due to bulky packaging and extended storage time cotton swabs become compressed and deformed. Furthermore, the steep difference in pricing between high quality cotton swabs and popular brands motivated the inventor to look for reliable ways to fluff the cotton-on-cotton swabs.

The present inventor realized in an inventive moment that inserting a needle gently into the cotton part of the cotton swab, and into several points, caused the cotton to increase in size, become more absorbent and did not severely damage the structure of cotton.

Furthermore, repeated rocking of needles inside the same points on the cotton part led to a more desirable results and a fluffier cotton swab.

Based on the issues raised the previous discussion, and the observations of the applicant, it is an object of the present invention to provide for a cotton swab fluffing device that will correct the deficiencies currently present in most sold cotton swabs.

Increasing the size of the fluff on a cotton swab while preserving the structural integrity of the cotton is another object of present invention.

Still another object of the present invention is to provide for a cotton swab fluffing device that is easy to use, successful in operation, aesthetically pleasing and can be mass produced at a reasonable price.

Cotton swabs are often used to remove unwanted or excess fluids such as blood from a minor injury, or smeared nail polish. Cotton swabs that are more absorbent are more desirable in such instances. Therefore, it is an object of present invention to provide a cotton swab fluffing device that makes the fluff on a cotton swab more absorbent.

Yet another object of present invention is to provide a cotton swab fluffing device that does not destroy the structure of the fluff and tear it apart.

In order to accomplish the objects described above, the present invention provides a rotating set of specially designed and configured needles attached to a set of rotating incomplete gears. Each of these incomplete gears is caused to rotate opposite to its adjacent incomplete gears causing the needles to engage the cotton swab from opposing sides and on multiple points.

The needles are configured to produce small holes homogeneously throughout the surface of the fluff causing it to become larger in size, homogeneous in shape and porous.

Furthermore, the configuration of the incomplete gears provides a path for the needles to engage the fluff without engaging the stick of the cotton swab. This design serves to preserve the adhesion of cotton to the surface stick and protect the needles from being damaged or causing damage to the sticks.

The present invention is designed and configured to accommodate standard cotton swabs regardless of their length or the material used in their sticks (plastic, paper or wooden sticks).

The foregoing has outlined Some of the more pertinent objects of the invention. These objects should be construed to be merely illustrative of some of the more prominent features and applications of the intended invention. Many other beneficial results can be obtained by applying the disclosed invention in a different manner or modifying the invention within the Scope of the disclosure. Accordingly, a fuller understanding of the invention may be had by referring to the detailed description of the preferred embodiments in addition to the scope of the invention defined by the claims taken in conjunction with the accompanying drawings.

In one embodiment of this invention the cotton swab fluffing device may be of a cylindrical shape but may also be of various other shapes or designs that render it small and compact and able to be hosted in numerous articles such as bathroom cabinets, first aid kits and makeup kits.

Most components of the present example of the invention may be manufactured of material that is light weight, durable and lends itself to low-cost manufacturing methods that are well known in the art, such as plastic inject molding, or plastic vacuum forming. Exceptions may include a plurality of spiral springs 201 and springs with hooks 104 that are common in the art. Another exception may be a plurality of incomplete gears 107, these may be made of metal, such as aluminum, stainless steel or carbon steel. They may also be made from durable plastic material that exhibits proper tensile strength and resiliency.

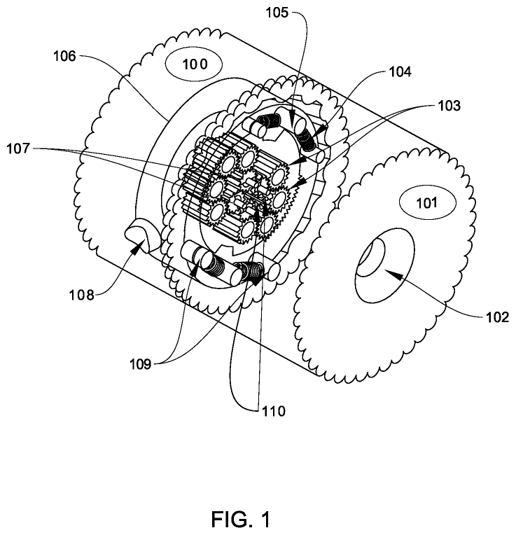

FIG. 1 is a sectioned rear perspective view of the cotton swab fluffing device showing various components according to an embodiment of present invention. A cotton swab may be inserted into an open end of a channel 102 in a non-rotatable connector 101 of the device. A rotatable housing connector 100 is then turned (approximately half circle) clockwise and counterclockwise (not necessarily in that order).

The channel 102 leads to an internal housing volume where the incomplete gears 107 are aligned around a circular space to host the cotton swab end. In this preferred example the top portions of the incomplete gears 107 are longitudinally cut, and a plurality of sharp needles 110 is aligned laterally in two or more rows on the surface of the cut part.

The channel 102 may be of a cylindrical shape that can accommodate the size of an enlarged (fluffed) cotton swab upon its removal from the device. The channel 102 may also feature a wide opening to allow for an easier insertion of the cotton swab end into the device. This wide opening may be of various shapes, for example, semispherical or cone shaped.

In this example, an internal gear wheel 106 housed within the rotatable housing connector 100 turns with the turning of that rotatable housing connector 100 for a calculated distance.

In an embodiment of present invention, the internal gear wheel 106 may comprise two or more holes on opposite sides of its outer wall, each hole houses a spiral spring 201 which urges a snaphead 108 against the interior wall of the rotatable housing connector. And in this example, the interior wall of the internal gear wheel 106 may also feature a pair of locking studs 400 on opposing sides. These locking studs 400 may be of any proper shape to fit into the tracks allotted for them and cause the required cessation of rotation for the internal gear wheel 106.

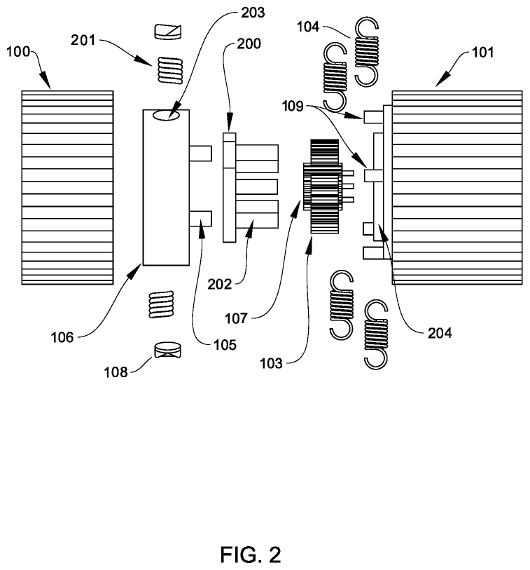

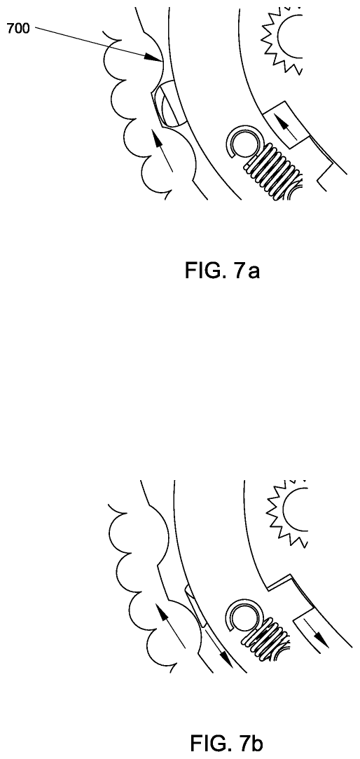

In one embodiment of present invention the interior wall of the rotatable housing connector 100 may be uniformly dotted with a plurality of notches 700 that are spaced uniformly. The notches 700 together with the spiral springs 201 and the snapheads 108 comprise a detent mechanism (as illustrated in FIG. 2, FIG. 7a and FIG. 7b)

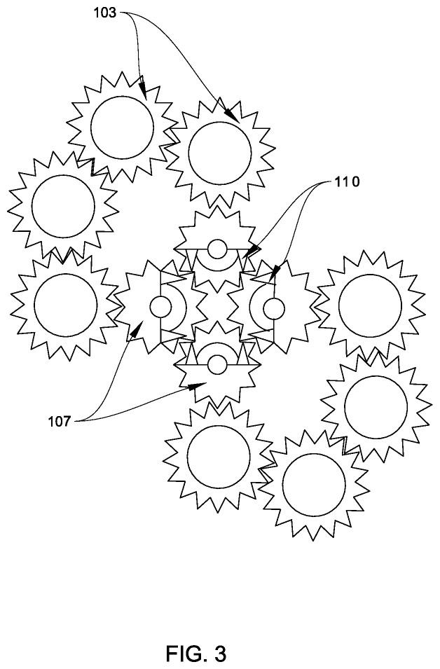

In an embodiment of present device, a plurality of gears 103 may operably connect the incomplete gears 107 to the internal gear wheel 106. Gear teeth 402 on the internal gear wheel 106 cause the gears 103 to rotate as the internal gear wheel 106 rotates. FIG. 3 depicts the alignment of the gears and incomplete gears in this embodiment.

In one example of current invention the gears 103 and the incomplete gears 107 may be aligned on an assembly plate 200. The gears 103 may be assembled on cylindrical posts 202, while the bottom ends of the incomplete gears 107 may be accommodated in holes on the assembly plate 200.

In an embodiment of current invention, the non-rotatable connector 101 may comprise a plate 204 with a mirror image perimeter to the assembly plate 200. This plate 204 may comprise holes 600 to accommodate the free ends of the cylindrical posts 202, and other holes 601 to accommodate the top ends of the incomplete gears 107.

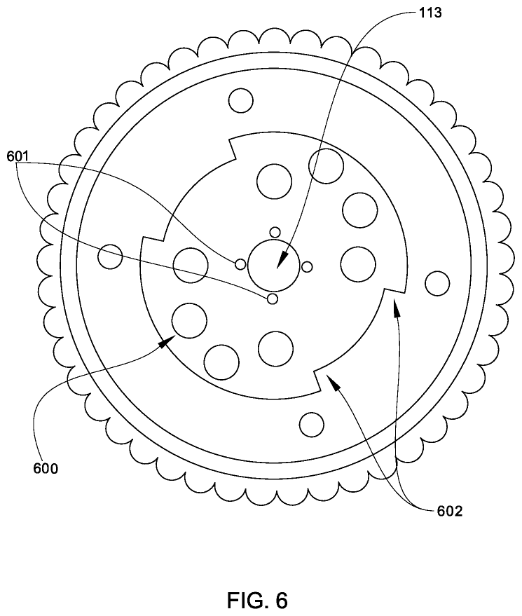

Two calculated slots are featured laterally along the perimeters of the assembly plate 200 and the plate 204 on the non-rotatable connector (refer to FIG. 6) These slots provide a path or a track 401 and 602 for the locking studs 400.

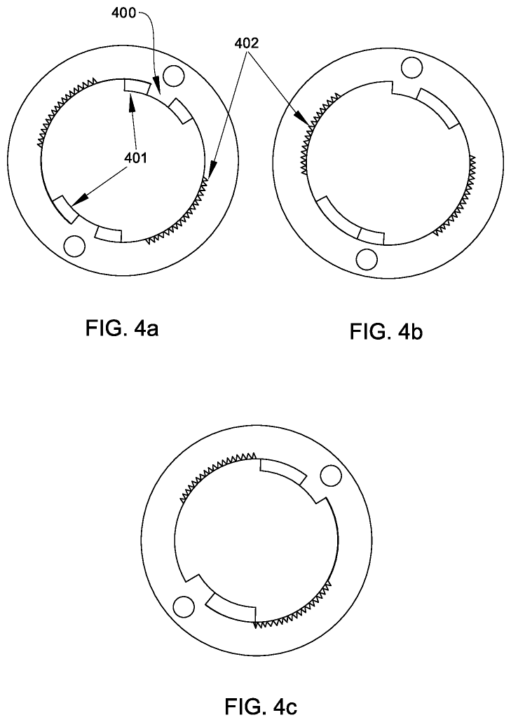

Once the swab fluffing device is in operation, and the rotatable housing connector 100 is being turned, the spiral springs 201 keep the snapheads 108 in the space (valley) between the notches 700, which ensures the rotation of the internal gear wheel 106 until the locking studs 400 reach the end of the tracks 401 and 602. At this point, the rotation of the internal gear wheel 106 is arrested, and the notches 700 begin to compress the snapheads 108. In this example, when the snapheads 108 become fully compressed under the peak of the notches 700 a plurality of springs with hooks 104 rushes them (the snapheads) into the next available space between the notches 700. This causes a temporary partial reversal of the rotation of the internal gear wheel 106. This reversal of rotation ends when the snapheads 108 are fully released back into the space between notches 700 and are caught by the side of next notch 700 to bring the internal gear wheel 106 back to the end of the tracks 401, and 602. (Refer to FIG. 4a and FIG. 4b and FIG. 4c)

In the example mentioned in the previous paragraph multiple notches 700 may pass over the snapheads 108 when the rotatable housing connector 100 is turned half a circle. This causes the internal gear wheel 106 to rock (vibrate) back and forth repeatedly around its center.

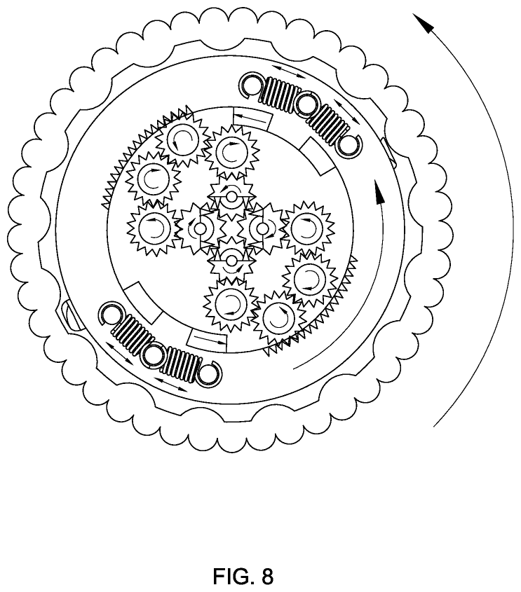

Since, the gears 103 and incomplete gears 107 are operably connected to each other's (FIG. 3) and since the movement of the gears 103 is dictated by the rotation of the internal gear wheel 106, any reversal of rotation on the internal gear wheel 106 is transmitted to the incomplete gears 107 (see FIG. 8) Consequently, in this example the rocking of the internal gear wheel 106 produces a rocking of the incomplete gears 107.

FIG. 8 is an illustration of the movement of the various parts in a counterclockwise rotation of the rotatable housing connector 100. While FIG. 7a and FIG. 7b are depictions of a detent mechanism during a clockwise rotation of the rotatable housing connector 100 in a present embodiment of the invention.

Also in this example, when the locking studs 400 reach the ends of the tracks 401 and 602 the needles 110 are fully inserted into the cotton part of the cotton swab. And when the incomplete gears 107 rock back and forth (vibrate) around their centers, the needles 110 vibrate within the cotton fluff (moving from ‘fully inserted’ to ‘partially out’ positions) causing the cotton on the cotton swab to increase in size (fluffing).

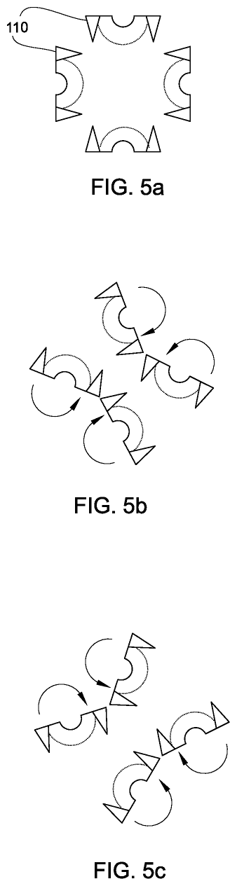

FIG. 5a depicts the plurality of needles 110 in the idle position. FIG. 5b and FIG. 5c illustrate the rotation of the same needles 110 during a clockwise and a counterclockwise turning of the rotatable housing connector 100, respectively, according to one embodiment of the cotton swab fluffing device. The rotation of opposing sides of the incomplete gears 107 and the engagement of needles 110 from these sides provides a firm grip on the cotton swab and prevents it from moving within the space allotted in the center of the device.

It is worth mentioning that although in this example the needles are cone shaped it should not be construed as a limitation of the invention. Other characterizations for the shape and the surface of the needles 110 may be possible and may be considered according to one or more embodiments of the present invention. For example, the needles may be arched to allow for a smoother penetration of cotton, and small bulge may be added on the surface of each needle 110 to further maximize the size of the stabbing holes in the cotton, and the surface roughness of the needles may be altered to achieve proper grip once these needles 110 are inside the cotton. The inventor chooses to represent a cone shaped needles in a preferred embodiment to simplify the description.

It will be apparent to one with skill in the art that the cotton swab fluffing device of the invention may be provided using some or all of the mentioned features and components without departing from the spirit and scope of the present invention. It will also be apparent to the skilled artisan that the embodiments described above are specific examples of a single broader invention which may have greater scope than any of the singular descriptions taught. There may be many alterations made in the descriptions without departing from the spirit and scope of the present invention.

What is claimed is:

1. A fluffing device for cotton swabs, comprising:
a first body having a central opening admitting a cotton end on a rod of a cotton swab;
geared elements within the body engaged to rotate relative to one another;
needles attached to individual ones of the geared elements, the needles positioned to penetrate the cotton end of the cotton swab with the swab inserted into the central opening; and
a physical input constrained to rotate the geared elements back and forth through a limited arc;
wherein the geared elements rotating move the needles in a manner urging portions of the cotton swab away from the rod, expanding and fluffing the cotton end.

2. The fluffing device of claim 1 wherein the physical input has a second body engaging the first body rotationally, relative rotation between the first and the second body rotating the geared elements.

3. The fluffing device of claim 2 wherein the first and the second body are cylindrical bodies rotating on a common axis.

4. The fluffing device of claim 3 wherein the relative rotation of the cylindrical bodies is opposed in both rotary directions by spring elements.

5. The fluffing device of claim 1 wherein the needles are conical in shape.

6. The fluffing device of claim 1 wherein the needles vary in diameter along a length.

7. The fluffing device of claim 1 wherein the needles have surface roughness.

8. The fluffing device of claim 3 wherein an outside surface of one or both of the first and the second body are roughened or corrugated improving grip.

9. The fluffing device of claim 1 wherein the needles are implemented on the gears in a manner to penetrate the cotton end from different directions.

10. The fluffing device of claim 1 wherein the central opening is larger in diameter than the cotton end of the cotton swab.

11. A method for fluffing a cotton swab, comprising:
inserting a cotton end on a rod of a cotton swab into a central opening of a first body of a fluffing device;
penetrating the cotton end by needles attached to individual ones of geared elements operating in a second body of the fluffing device, the geared elements engaged to rotate relative to one another; and
rotating a physical input constrained to rotate the geared elements back and forth through a limited arc, the rotation of the geared elements manipulating the needles in a manner urging portions of the cotton swab away from the rod, expanding and fluffing the cotton end.

12. The method of claim 11 comprising engaging the first body by a second body rotationally, relative rotation between the first and the second body rotating the geared elements.

13. The method of claim 12 comprising rotating the first and the second cylindrical bodies on a common axis.

14. The method of claim 13 comprising opposing the relative rotation of the cylindrical bodies in both rotary directions by spring elements.

15. The method of claim 11 comprising implementing the needles as conical in shape.

16. The method of claim 11 comprising implementing the needles to vary in diameter along a length.

17. The method of claim 11 comprising implementing the needles with surface roughness.

18. The method of claim 13 comprising implementing an outside surface of one or both of the first and the second body as roughened or corrugated, improving grip.

19. The method of claim 1 comprising implementing the needles on the gears in a manner to penetrate the cotton end from different directions.

20. The method of claim 11 comprising making the central opening larger in diameter than the cotton end of the cotton swab.
